SEQUENCE LISTING (4 sequences)

SEQ ID NO: 1 - protein, 568 aa, Homo sapiens
MVLLHWCLLWLLFPLSSRTQKLPTRDEELFQMQIRDKAFFHDSSVIPDGAEISSYLFRDTPKRYFFVVEEDNTPLSVTVTPCDAPLEWKLSLQELPEDRSGEGSGDLEPLEQQKQQIINEEGTELFSYKGNDVEYFISSSSPSGLYQLDLLSTEKDTHFKVYATTTPESDQPYPELPYDPRVDVTSLGRTTVTLAWKPSPTASLLKQPIQYCVVINKEHNFKSLCAVEAKLSADDAFMMAPKPGLDFSPFDFAHFGFPSDNSGKERSFQAKPSPKLGRHVYSRPKVDIQKICIGNKNIFTVSDLKPDTQYYFDVFVVNINSNMSTAYVGTFARTKEEAKQKTVELKDGKITDVFVKRKGAKFLRFAPVSSHQKVTFFIHSCLDAVQIQVRRDGKLLLSQNVEGIQQFQLRGKPKAKYLVRLKGNKKGASMLKILATTRPTKQSFPSLPEDTRIKAFDKLRTCSSATVAWLGTQERNKFCIYKKEVDDNYNEDQKKREQNQCLGPDIRKKSEKVLCKYFHSQNLQKAVTTETIKGLQPGKSYLLDVYVIGHGGHSVKYQSKVVKTRKFC

SEQ ID NO: 2 - protein, 15 aa, synthetic construct
TCSSATVAWLGTQER

SEQ ID NO: 3 - protein, 15 aa, synthetic construct
REQTGLWAVTASSCT

SEQ ID NO: 4 - protein, 13 aa, synthetic construct
SSATVAWLGTQER

(19) United States
(12) Patent Application Publication
(10) Pub. No.: US 2024/0245712 A1
(43) Pub. Date: Jul. 25, 2024

(54) METHODS AND MATERIALS FOR IDENTIFYING AND TREATING MEMBRANOUS NEPHROPATHY

(71) Applicant: Mayo Foundation for Medical Education and Research, Rochester, MN (US)

(72) Inventors: Sanjeev Sethi, Rochester, MN (US); Fernando C. Fervenza, Rochester, MN (US); Benjamin J. Madden, Stewartville, MN (US)

(21) Appl. No.: 18/413,949

(22) Filed: Jan. 16, 2024

Related U.S. Application Data
(60) Provisional application No. 63/440,272, filed on Jan. 20, 2023.

(51) Int. Cl. A61K 31/65; A61K 31/43; A61P 13/12; G01N 33/564

(52) U.S. Cl. CPC A61K 31/65; A61K 31/43; A61P 13/12; G01N 33/564; G01N 2800/347

(57) ABSTRACT

This document relates to methods and materials involved in identifying and/or treating mammals having membranous nephropathy (e.g., membranous nephropathy with an elevated level of a neuron-derived neurotrophic factor (NDNF) polypeptide in the glomerular basement membrane (GBM)). For example, methods and materials for administering one or more antimicrobial agents to treat a mammal (e.g., a human) having membranous nephropathy are provided.

### CROSS-REFERENCE TO RELATED APPLICATIONS

This application claims the benefit of U.S. Patent Application Ser. No. 63/440,272, filed on Jan. 20, 2023. The disclosure of the prior application is considered part of, and is incorporated by reference in, the disclosure of this application.

### SEQUENCE LISTING

This application contains a Sequence Listing that has been submitted electronically as an XML file named “07039-2186001_SL.xml.” The XML file, created on Dec. 20, 2023, is 5000 bytes in size. The material in the XML file is hereby incorporated by reference in its entirety.

### TECHNICAL FIELD

This document relates to methods and materials involved in identifying and/or treating mammals having membranous nephropathy (e.g., membranous nephropathy with an elevated level of a neuron-derived neurotrophic factor (NDNF) polypeptide in the glomerular basement membrane (GBM)). For example, this document provides methods and materials for administering one or more antimicrobial agents to treat a mammal (e.g., a human) having membranous nephropathy.

### BACKGROUND INFORMATION

Syphilis is caused by the bacteria Treponema pallidum and is one of the most common sexually transmitted diseases (Hook, The Lancet, 389:1550-1557 (2017)). More than 5 million cases of syphilis are diagnosed worldwide each year. The incidence of syphilis is on the rise in the United States with the CDC reporting a 28% increase in 2021 compared to 2017. 171,024 cases of syphilis were reported in 2021, up from 101,590 cases in 2017 (Dyer, BMJ, 378:o2355 (2022); and CDC—National Center for Health Statistics—Preliminary 2021 STD Surveillance Data, www.cdc.gov/std/statistics/2021/default.htm.: September 2022 ed). Furthermore, cases of late syphilis have also risen substantially (Dyer, BMJ, 378:o2355 (2022)). Multisystemic involvement occurs in late syphilis (secondary and tertiary syphilis) and in addition to skin lesions patients may present with lymphadenopathy, hepatosplenomegaly, hepatitis and nephrotic syndrome (Hook, The Lancet, 389:1550-1557 (2017)).

Membranous nephropathy results from subepithelial deposition of immune complexes along the GBM. Membranous nephropathy is often classified into primary membranous nephropathy, where there is no identifiable underlying disease association, and secondary membranous nephropathy, where membranous nephropathy may be associated with an autoimmune disease, infection, malignancy, hematopoietic stem cell transplant, etc. (Beck et al., J. Clin. Invest., 124:2307-2314 (2014); Ronco et al., The Lancet, 385:1983-1992 (2015); and Couser, Clin. J. Am. Soc. Nephrol., 12:983-997 (2017)). Target antigens for many types of membranous nephropathy have been identified (Beck et al., New Eng. J. Med., 361:11-21 (2009); Tomas et al., New Eng. J. Med, 371:2277-2287 (2014); Sethi, J. Am. Soc. Nephrol., 32:268 (2021); Sethi et al., J. Am. Soc. Nephrol, 30:1123-1136 (2019); Sethi et al., Kidney Int., 97:163-174 (2020); Sethi et al., Kidney Int., 98:1253-1265 (2020); Sethi et al., J. Am. Soc. Nephrol., 32:1249-1261 (2021); Caza et al., Kidney Int., 100:171-181 (2020); and Al-Rabadi et al., J. Am. Soc. Nephrol., 32:1666-1681 (2021)).

Membranous nephropathy is the most common cause of nephrotic syndrome in syphilis (Shettigar et al., Intern. Med. J., 51:1160-1167 (2021); Araújo Sde et al., Braz. J. Infect. Dis., 19:442-443 (2015); Wanderley et al., Clin. Kidney J., 14:1857-1858 (2021); Scaperotti et al., BMJ Case Rep., 14 (2021); Satoskar et al., Am. J. Kidney Dis., 55:386-390 (2010); and Hunte et al., J. Am. Soc. Nephrol., 3:1351-1355 (1993)). The target antigen(s) in syphilis-associated membranous nephropathy remain elusive.

### SUMMARY

This document provides methods and materials involved in identifying and/or treating mammals (e.g., humans) having membranous nephropathy (e.g., membranous nephropathy with an elevated level of a NDNF polypeptide in the GBM). For example, this document provides methods and materials for identifying a mammal (e.g., a human) having membranous nephropathy as having an elevated level of a NDNF polypeptide in the GBM that can serve as a target antigen in membranous nephropathy. This document also provides methods and materials for identifying a mammal (e.g., a human) having membranous nephropathy as having the presence of autoantibodies having binding specificity for a NDNF polypeptide. As described herein, mammals (e.g., humans) having membranous nephropathy can be identified as having an elevated level of a NDNF polypeptide in the GBM. In such cases, the mammal can be classified as having a form of membranous nephropathy that includes an elevated level of a NDNF polypeptide in the GBM. As also described herein, mammals (e.g., humans) having membranous nephropathy can be identified as having autoantibodies having binding specificity for a NDNF polypeptide. In such cases, the mammal can be classified as having a form of membranous nephropathy that includes the presence of autoantibodies having binding specificity for a NDNF polypeptide. Identifying mammals (e.g., humans) as having membranous nephropathy that includes an elevated level of a NDNF polypeptide in the GBM and/or that includes the presence of autoantibodies having binding specificity for a NDNF polypeptide can allow clinicians and patients to proceed with appropriate membranous nephropathy treatment options.

This document also provides methods and materials for treating membranous nephropathy. For example, a mammal (e.g., a human) having membranous nephropathy that was identified as having an elevated level of a NDNF polypeptide in the GBM, as having autoantibodies having binding specificity for a NDNF polypeptide, or as having both an elevated level of a NDNF polypeptide in the GBM and autoantibodies having binding specificity for a NDNF polypeptide can be administered one or more antimicrobial agents (e.g., one or more antibiotic agents) to reduce infection (and subsequent inflammation) and/or B-cell autoantibody production. As described herein, mammals (e.g., humans) having membranous nephropathy and identified as having an elevated level of a NDNF polypeptide in the GBM and/or as having autoantibodies having binding specificity for a NDNF polypeptide have a form of membranous nephropathy that is caused by the presence of antigen-autoantibody complexes where the antigen is a NDNF polypeptide. In such cases, the mammal (e.g., human) can be effectively treated using one or more antimicrobial agents (e.g., one or more antibiotic agents) to reduce infection (and subsequent inflammation) and/or B-cell autoantibody production. Having the ability to administer one or more antimicrobial agents to mammals (e.g., humans) (a) having membranous nephropathy and (b) identified as having an elevated level of a NDNF polypeptide in the GBM and/or as having autoantibodies having binding specificity for a NDNF polypeptide can allow clinicians and patients to treat membranous nephropathy effectively.

In some cases, identification of the target antigen and autoantibodies can be used to diagnosis and/or to manage treatment of a mammal (e.g., a human) with membranous nephropathy. For example, a mammal (e.g., a human) having membranous nephropathy (e.g., membranous nephropathy having an elevated level of a NDNF polypeptide in the GBM and/or having autoantibodies having binding specificity for a NDNF polypeptide) can be administered one or more antimicrobial agents (e.g., one or more antibiotic agents) to treat membranous nephropathy. In some cases, the response to the antimicrobial treatment can be monitored for a decrease or complete elimination of the autoantibodies having binding specificity for a NDNF polypeptide. In some cases, the response to treatment can be monitored by examining a kidney biopsy for a decrease or elimination of a NDNF polypeptide. In some cases, a mammal (e.g., a human) having membranous nephropathy can be administered one or more antimicrobial agents (e.g., one or more antibiotic agents) to treat membranous nephropathy based on the presence of an autoantibody having binding specificity for a NDNF polypeptide in the absence of evaluating a kidney biopsy for an elevated level of a NDNF polypeptide. Although kidney biopsies showing an accumulation of a NDNF polypeptide in GBM may be considered an effective manner for diagnosis of membranous nephropathy, the presence of autoantibodies having binding specificity for a NDNF polypeptide can be used to identify membranous nephropathy associated with accumulation of NDNF polypeptides without the need for performing a kidney biopsy.

Also, having the ability to identify a mammal as having membranous nephropathy based, at least in part, on (a) having an elevated level of a NDNF polypeptide in the GBM and/or (b) having autoantibodies having binding specificity for a NDNF polypeptide provides a unique and unrealized opportunity to provide a safe and effective use of one or more antimicrobial agents (e.g., one or more antibiotic agents) to treat membranous nephropathy in this identified patient population.

In general, one aspect of this document features methods for identifying a mammal having syphilis as having membranous nephropathy having an elevated level of a NDNF polypeptide within kidney tissue of the mammal. The methods can include, or consist essentially of, (a) determining the presence or absence of autoantibodies specific for the NDNF polypeptide within a sample obtained from a mammal, (b) classifying the mammal as having the membranous nephropathy if the autoantibodies are present within the sample, and (c) classifying the mammal as not having the membranous nephropathy if the autoantibodies are absent within the sample. The mammal can be a human. The sample can be a blood sample. The membranous nephropathy can lack an elevated level of a protocadherin-7 (PCDH7) polypeptide, a semaphorin-3B (SEMA3B) polypeptide, a neural epidermal growth factor (EGF)-like 1 (NELL-1) polypeptide, an exostosin 1 (EXT1) polypeptide, an exostosin 2 (EXT2) polypeptide, a PLA2R polypeptide, a THSD7A polypeptide within the kidney tissue, or a protocadherin FAT1 (FAT1) polypeptide. The membranous nephropathy can lack an elevated level of a PCDH7 polypeptide, a SEMA3B polypeptide, a NELL-1 polypeptide, an EXT1 polypeptide, an EXT2 polypeptide, a PLA2R polypeptide, a THSD7A polypeptide, and a FAT1 polypeptide within the kidney tissue. The method can include detecting the presence of the autoantibodies within the sample and classifying the mammal as having the membranous nephropathy. The method can include detecting the absence of the autoantibodies within the sample and classifying the mammal as not having the membranous nephropathy.

In another aspect, this document features methods for identifying a mammal having syphilis as having membranous nephropathy having kidney tissue having an elevated level of a NDNF polypeptide. The methods can include, or consist essentially of, (a) determining the presence or absence of the kidney tissue within a sample obtained from a mammal, (b) classifying the mammal as having the membranous nephropathy if the presence is determined, and (c) classifying the mammal as not having the membranous nephropathy if the absence is determined. The mammal can be a human. The kidney tissue can lack an elevated level of a PCDH7 polypeptide, a SEMA3B polypeptide, a NELL-1 polypeptide, an EXT1 polypeptide, an EXT2 polypeptide, a PLA2R polypeptide, a THSD7A polypeptide, or a FAT1 polypeptide. The kidney tissue can lack an elevated level of a PCDH7 polypeptide, a SEMA3B polypeptide, a NELL-1 polypeptide, an EXT1 polypeptide, an EXT2 polypeptide, a PLA2R polypeptide, a THSD7A polypeptide, and a FAT1 polypeptide. The method can include detecting the presence and classifying the mammal as having the membranous nephropathy. The method can include detecting the absence and classifying the mammal as not having the membranous nephropathy.

In another aspect, this document features methods for identifying a mammal having syphilis as having membranous nephropathy having autoantibodies specific for a NDNF polypeptide. The methods can include, or consist essentially of, (a) determining the presence or absence of the autoantibodies within a sample obtained from a mammal having syphilis, (b) classifying the mammal as having the membranous nephropathy if the autoantibodies are present within the sample, and (c) classifying the mammal as not having the membranous nephropathy if the autoantibodies are absent within the sample. The mammal can be a human. The sample can be a blood sample. The kidney tissue of the mammal can lack an elevated level of a PCDH7 polypeptide, a SEMA3B polypeptide, a NELL-1 polypeptide, an EXT1 polypeptide, an EXT2 polypeptide, a PLA2R polypeptide, a THSD7A polypeptide, or a FAT1 polypeptide. The kidney tissue of the mammal can lack an elevated level of a PCDH7 polypeptide, a SEMA3B polypeptide, a NELL-1 polypeptide, an EXT1 polypeptide, an EXT2 polypeptide, a PLA2R polypeptide, a THSD7A polypeptide, and a FAT1 polypeptide. The method can include detecting the presence within the sample and classifying the mammal as having the membranous nephropathy. The method can include detecting the absence within the sample and classifying the mammal as not having the membranous nephropathy.

In another aspect, this document features methods for treating a mammal having membranous nephropathy. The methods can include, or consist essentially of, (a) identifying a mammal as having membranous nephropathy having (i) autoantibodies specific for a NDNF polypeptide or (ii) kidney tissue having an elevated level of the NDNF polypeptide, and (b) administering an antimicrobial agent to the mammal. The mammal can be a human. The mammal can be identified as having the membranous nephropathy having the autoantibodies. The mammal can be identified as having the membranous nephropathy having the kidney tissue. The antimicrobial agent can be an antibiotic agent. The antibiotic agent can be penicillin, doxycycline, tetracycline, or amoxicillin. The mammal can be administered an immunosuppressive agent. The level of autoantibodies present within the mammal can be reduced by at least 5 percent following the administering step. The level of autoantibodies present within the mammal can be reduced by at least 25 percent following the administering step. The level of autoantibodies present within the mammal can be reduced by at least 50 percent following the administering step.

In another aspect, this document features methods for treating a mammal having membranous nephropathy. The methods can include, or consist essentially of, administering an antimicrobial agent to a mammal identified as having membranous nephropathy having (i) autoantibodies specific for a NDNF polypeptide or (ii) kidney tissue having an elevated level of the NDNF polypeptide. The mammal can be a human. The mammal can have been identified as having the membranous nephropathy having the autoantibodies. The mammal can have been identified as having the membranous nephropathy having the kidney tissue. The antimicrobial agent can be an antibiotic agent. The antibiotic agent can be penicillin, doxycycline, tetracycline, or amoxicillin. The mammal can be administered an immunosuppressive agent. The level of autoantibodies present within the mammal can be reduced by at least 5 percent following the administering step. The level of autoantibodies present within the mammal can be reduced by at least 25 percent following the administering step. The level of autoantibodies present within the mammal can be reduced by at least 50 percent following the administering step.

In another aspect, this document features methods for treating a mammal having membranous nephropathy and kidney tissue having an elevated level of a NDNF polypeptide. The methods can include, or consist essentially of, administering an antimicrobial agent to a mammal having membranous nephropathy and kidney tissue having an elevated level of a NDNF polypeptide. The mammal can be a human. The mammal can have autoantibodies specific for the polypeptide. The mammal can have been identified as having the kidney tissue. The antimicrobial agent can be an antibiotic agent. The antibiotic agent can be penicillin, doxycycline, tetracycline, or amoxicillin. The mammal can be administered an immunosuppressive agent. The level of autoantibodies present within the mammal can be reduced by at least 5 percent following the administering step. The level of autoantibodies present within the mammal can be reduced by at least 25 percent following the administering step. The level of autoantibodies present within the mammal can be reduced by at least 50 percent following the administering step.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1. Discovery and validation MS/MS cohorts. Initial discovery cohort of 250 cases of PLA2R-negative membranous nephropathy (MN) revealed 3 cases of NDNF-associated MN. One patient had syphilis and hepatitis B. The syphilis status was not known in the remaining two cases. A validation cohort of 14 cases of hepatitis B-MN was negative for NDNF in all cases, while a validation cohort of five cases of syphilis-MN was positive for NDNF in all cases.

FIGS. 2A-2C. Proteomic Identification of NDNF in PLA2R-negative MN. Glomeruli were microdissected and analyzed using mass spectrometry. FIG. 2A) Detection of NDNF in 8 cases of PLA2R-negative MN (top row). Case 8 and 9 are the same patient who was biopsied twice. Numbers in boxes represent total spectral counts of MS/MS matches to a respective protein. All 9 biopsies showed high total spectral counts for NDNF and IgG1, IgG2, IgG3 subtypes. Baseline spectral counts of PLA2R were also detected in 8 of 9 biopsies. For comparison, the pooled total spectral counts from 6 control cases (time 0 protocol transplant biopsies) are also shown, NDNF was not present in the control cases. Case 10 is a patient with diffuse proliferative glomerulonephritis in a patient with active syphilis. FIG. 2B) Representative sequence (SEQ ID NO:1) coverage map of a NDNF polypeptide from 1 case. Amino acids highlighted and in bold letters are the amino acids detected. FIG. 2C) An example of MS/MS spectra match to a sequence from the NDNF polypeptide (SEQ ID NO:2 (top) and SEQ ID NO:3 (bottom)). Example MS/MS spectra of ion 833.90 [M+2H]²⁺⁺ matched to the NDNF peptide sequence SSATVAWLGTQER (SEQ ID NO:4).

FIGS. 3A-3B. Immunohistochemical (IHC) staining and confocal IF staining for NDNF. FIG. 3A) Discovery cohort. Panels A-B) IHC staining showing granular staining for NDNF along GBM (A-patient 3, B-patient 2). Panels C-H) Confocal IF (patient 1) showing NDNF (Panels C and F) and IgG (Panels D and G) and merged image (Panels E and H). Panels C-E show the entire glomerulus and Panels F-H show enlarged images of selected capillary loops to focus on the colocalization of NDNF and IgG. FIG. 3B) Validation cohort. Confocal microscopy of validation cohort (case 4-7). Each row is one case, top row=case 4, 2^{nd} row=case 5, 3^{rd} row=case 6, and 4^{th} row=case 7. First column is NDNF, second column shows IgG, 3^{rd} column shows an overlay of NDNF and IgG, and 4^{th} column shows a selected high power of NDNF-IgG overlay of few capillary loops. Note the IgG in serum staining as lakes within come capillary loops. Bottom row were control cases. A case of PLA2R-associated MN showed no staining for NDNF (column 1), granular IgG staining (column 2), and an overlay showed only IgG staining (column 3). An additional case of EXT1/2-associated MN also showed no staining for NDNF (column 4).

FIGS. 4A-4B. Western blot analysis showing IgG from eluate of NDNF-associated MN bind to non-reduced (FIG. 4A) and reduced (FIG. 4B) NDNF (2000 ng loaded in each lane). Control: non-reduced and reduced NDNF were detected by rabbit anti-human NDNF (1:1000) at approximately 44 kDa (arrow) (column 1 in FIGS. 4A and 4B). Eluate: non-reduced and reduced NDNF are detected using eluate from NDNF-associated MN using a secondary anti-human IgG (1:5000) (column 2 in FIGS. 4A and 4B). The binding was not detected using eluate from PLA2R-associated MN (column 3 in FIGS. 4A and 4B). Near infrared fluorescence was detected at the 700 and 800 nm channel in the Odyssey Infrared Imaging System (LI-COR® Biosciences, Lincoln, NE). The reading was done for 10 minutes in each channel (total 20 minutes).

FIG. 5. Representative kidney biopsy findings in NDNF-associated MN. Panel A) Light microscopy showing thickened glomerular capillary walls, with no proliferative features (periodic acid Schiff stain 40×). Panels B-H) Immunofluorescence microscopy showed bright granular capillary wall staining for IgG (Panel B), C3 (Panel C), IgG1 (Panel E), IgG2 (Panel F), and IgG3 (Panel G), but no staining for PLA2R (Panel D) or IgG4 (Panel H). Electron microscopy (Panels I-L) showed superficial hump like deposits with minimal or no glomerular basement membrane reaction in all cases (Panels I-J=case 3, Panel K=case 2, and Panel L=case 1). Panels A-J are from case 3.

FIG. 6. Representative electron microscopy images from validation cases showed subepithelial-hump like deposits.

### DETAILED DESCRIPTION

This document provides methods and materials for identifying and/or treating mammals (e.g., humans) having membranous nephropathy (e.g., membranous nephropathy with an elevated level of a NDNF polypeptide in the GBM). For example, this document provides methods and materials for identifying a mammal (e.g., a human) having membranous nephropathy as having (a) autoantibodies specific for a NDNF polypeptide and/or (b) a GBM having an elevated level of a NDNF polypeptide.

Any appropriate mammal having membranous nephropathy can be identified as having (a) autoantibodies specific for a NDNF polypeptide and/or (b) kidney tissue (e.g., GBM) having an elevated level of a NDNF polypeptide. In some cases, a mammal having membranous nephropathy also can have an infection (e.g., a bacterial infection). For example, a mammal having membranous nephropathy also can have an infection associated membranous nephropathy. Examples of infections that can be associated with membranous nephropathy identified as having (a) autoantibodies specific for a NDNF polypeptide and/or (b) kidney tissue (e.g., GBM) having an elevated level of a NDNF polypeptide include, without limitation, T. pallidum infections (e.g., T. pallidum infections causing syphilis). In some cases, a mammal having membranous nephropathy can have syphilis-associated membranous nephropathy. Examples of mammals having membranous nephropathy that can be identified as having (a) autoantibodies specific for a NDNF polypeptide and/or (b) kidney tissue (e.g., GBM) having an elevated level of a NDNF polypeptide as described herein include, without limitation, primates (e.g., humans and monkeys), dogs, cats, horses, cows, pigs, sheep, rabbits, mice, and rats. For example, humans having membranous nephropathy can be identified as having (a) autoantibodies specific for a NDNF polypeptide and/or (b) kidney tissue such as GBM having an elevated level of a NDNF polypeptide as described herein.

Any appropriate method can be used to determine if a mammal (e.g., a human) has autoantibodies specific for a NDNF polypeptide. For example, immunological assays using a NDNF polypeptide (or a fragment thereof capable of binding to an anti-NDNF antibody) can be used to determine if a sample contains autoantibodies specific for a NDNF polypeptide. In some cases, an immobilized NDNF polypeptide (or an immobilized fragment thereof) can be used to capture an anti-NDNF autoantibody if present within a sample being tested, and an anti-Ig antibody (e.g., an anti-human IgG antibody when testing for human autoantibodies) can be used to determine whether or not autoantibodies were captured. In some cases, an anti-Ig antibody can be labeled (e.g., fluorescently or enzymatically labeled) to aid in detection. Any appropriate sample can be used to determine if a mammal (e.g., a human) has autoantibodies specific for a NDNF polypeptide. For example, blood samples (e.g., whole blood samples, serum samples, and plasma samples) or urine samples obtained from a mammal being tested can be used to determine if a mammal (e.g., a human) has autoantibodies specific for a NDNF polypeptide.

Any appropriate method can be used to determine if a mammal (e.g., a human) has kidney tissue (e.g., GBM) having an elevated level of a NDNF polypeptide. For example, immunological techniques such as IHC techniques, immunofluorescence (IF) techniques, or western blot techniques can be used to determine if a mammal (e.g., a human) has kidney tissue (e.g., GBM) having an elevated level of a NDNF polypeptide. In some cases, a kidney tissue sample obtained from a mammal to be tested can be stained using an anti-NDNF antibody to determine if the mammal has kidney tissue (e.g., GBM) having an elevated level of NDNF polypeptides. Any appropriate sample can be used to determine if a mammal (e.g., a human) has kidney tissue (e.g., GBM) having an elevated level of a NDNF polypeptide. For example, kidney tissue biopsies can be obtained from a mammal (e.g., a human) being tested and used to determine if the mammal (e.g., the human) has kidney tissue (e.g., GBM) having an elevated level of a NDNF polypeptide.

The term “elevated level” as used herein with respect to a NDNF polypeptide present within kidney tissue (e.g., GBM) refers to a level of NDNF polypeptides present within kidney tissue (e.g., GBM) that is greater (e.g., at least 10, 25, 35, 45, 50, 55, 65, 75, 80, 90, or 100 percent greater) than the median level of NDNF polypeptides present within normal kidney tissue (e.g., a normal GBM) of comparable mammals not having membranous nephropathy.

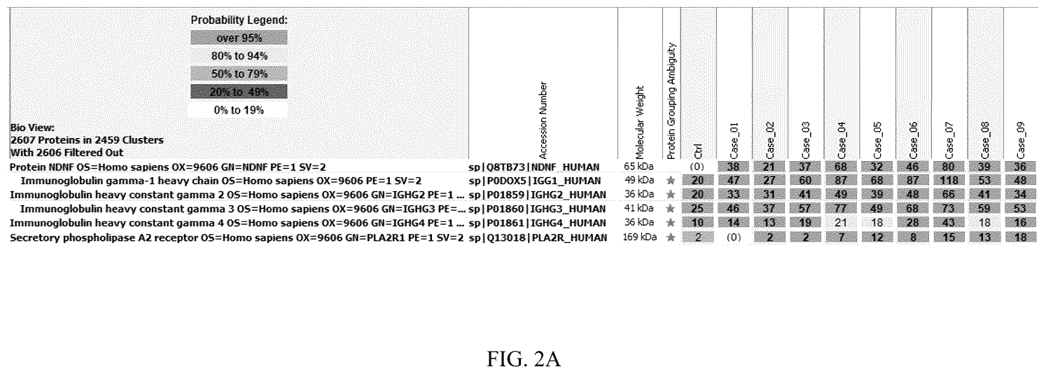

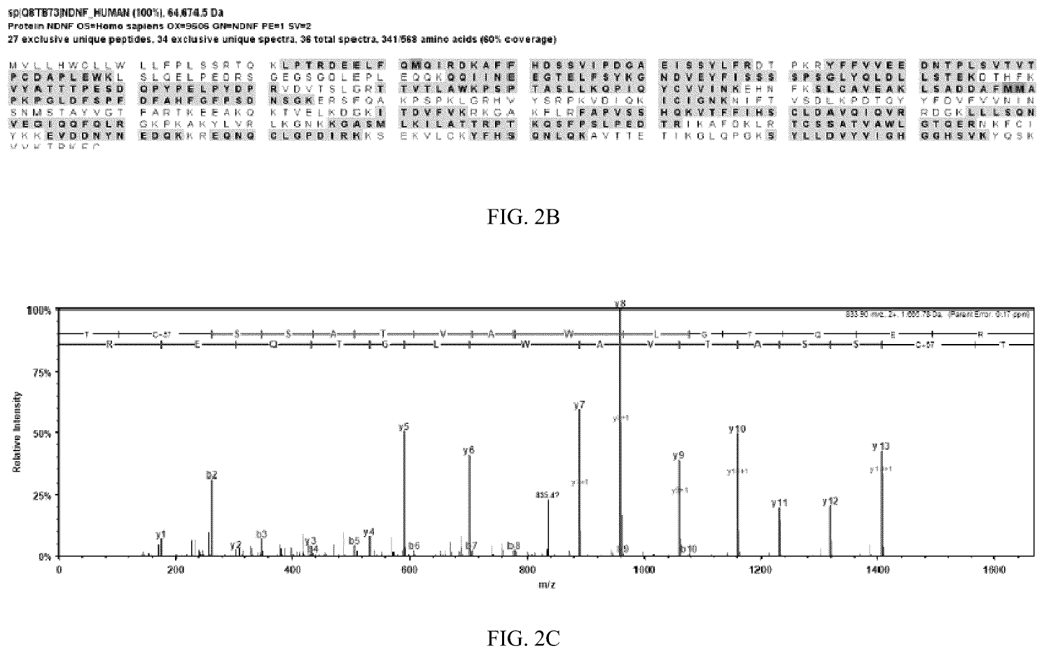

A NDNF polypeptide can include any appropriate amino acid sequence. Examples of human NDNF polypeptides include, without limitation, those polypeptides having the amino acid sequence set forth in SEQ ID NO:1 (see, e.g., FIG. 2B). In some cases, the amino acid sequence of a NDNF polypeptide can have a sequence that deviates from the nucleotide sequence set forth in SEQ ID NO:1, sometimes referred to as a variant sequence. For example, a NDNF polypeptide can have an amino acid sequence that includes one or more modifications (e.g., deletions, insertions, and substitutions) to the amino acid sequence set forth in SEQ ID NO:1. For example, an amino acid sequence of a NDNF polypeptide can have at least 80% sequence identity (e.g., about 82% sequence identity, about 85% sequence identity, about 88% sequence identity, about 90% sequence identity, about 93% sequence identity, about 95% sequence identity, about 97% sequence identity, about 98% sequence identity, or about 99% sequence identity) to the amino acid sequence set forth in SEQ ID NO:1. Percent sequence identity is calculated by determining the number of matched positions in aligned amino acid sequences, dividing the number of matched positions by the length of an aligned amino acid sequence, and multiplying by 100. A matched position refers to a position in which identical amino acids occur at the same position in aligned amino acid sequences. Percent sequence identity also can be determined for any nucleic acid sequence.

The percent sequence identity between a particular nucleic acid or amino acid sequence and a sequence referenced by a particular sequence identification number (e.g., SEQ ID NO:1) is determined as follows. First, an amino acid sequence is compared to the sequence set forth in a particular sequence identification number using the BLAST 2 Sequences (Bl2seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the Bl2seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq-i c:\seq1.txt-j c:\seq2.txt-p blastp-o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical amino acid residue is presented in both sequences. The percent sequence identity is determined by dividing the number of matches by the length of the sequence set forth in the identified sequence (e.g., SEQ ID NO:1), followed by multiplying the resulting value by 100. For example, an amino acid sequence that has 340 matches when aligned with the sequence set forth in SEQ ID NO: 1 is 95.5 percent identical to the sequence set forth in SEQ ID NO:1 (i.e., 340÷356×100=95.5056). It is noted that the percent sequence identity value is rounded to the nearest tenth. For example, 75.11, 75.12, 75.13, and 75.14 is rounded down to 75.1, while 75.15, 75.16, 75.17, 75.18, and 75.19 is rounded up to 75.2. It also is noted that the length value will always be an integer.

In some cases, a human NDNF polypeptide can have the amino acid sequence set forth in FIG. 2B.

Once a mammal (e.g., a human) having membranous nephropathy is identified as having autoantibodies specific for a NDNF polypeptide as described herein, the mammal can be classified as having membranous nephropathy that includes the presence of those autoantibodies (e.g., membranous nephropathy that includes the presence of anti-NDNF autoantibodies). In some cases, a mammal (e.g., a human) having membranous nephropathy that is identified as having autoantibodies specific for a NDNF polypeptide as described herein can be classified as having membranous nephropathy that includes kidney tissue having an elevated level of NDNF polypeptides.

Once a mammal (e.g., a human) having membranous nephropathy is identified as having kidney tissue (e.g., GBM) having an elevated level of a NDNF polypeptide as described herein, the mammal can be classified as having membranous nephropathy that includes the presence of that kidney tissue (e.g., membranous nephropathy that includes the presence of kidney tissue such as GBM having an elevated level of NDNF polypeptides). In some cases, a mammal (e.g., a human) having membranous nephropathy that is identified as having kidney tissue (e.g., GBM) having an elevated level of a NDNF polypeptide as described herein can be classified as having membranous nephropathy that includes autoantibodies specific for a NDNF polypeptide.

As described herein, this document also provides methods and materials for treating a mammal having membranous nephropathy. For example, a mammal (e.g., a human) having membranous nephropathy that is identified as having (a) autoantibodies specific for a NDNF polypeptide and/or (b) kidney tissue (e.g., GBM) having an elevated level of a NDNF polypeptide as described herein can be treated with one or more antimicrobial agents. In some cases, a mammal (e.g., a human) having membranous nephropathy that is identified as having (a) autoantibodies specific for a NDNF polypeptide and/or (b) kidney tissue (e.g., GBM) having an elevated level of a NDNF polypeptide as described herein can be administered, or instructed to self-administer, one or more antimicrobial agents to treat membranous nephropathy.

Any appropriate antimicrobial agent can be administered to a mammal (e.g., a human that was identified as having (a) autoantibodies specific for a NDNF polypeptide and/or (b) kidney tissue (e.g., GBM) having an elevated level of a NDNF polypeptide as described herein) to treat membranous nephropathy. In some cases, an antimicrobial agent used as described herein to treat membranous nephropathy can be an antibiotic agent. Examples of antimicrobial agents that can be used as described herein to treat membranous nephropathy include, without limitation, penicillin, doxycycline, tetracycline, and amoxicillin.

In some cases, one or more antimicrobial agents can be administered to a mammal once or multiple times over a period of time ranging from days to months. In some cases, one or more antimicrobial agents can be given to achieve remission of membranous nephropathy, and then given during follow up periods to prevent relapse of the membranous nephropathy.

In some cases, one or more antimicrobial agents can be formulated into a pharmaceutically acceptable composition for administration to a mammal (e.g., a human) having membranous nephropathy to reduce infection (and subsequent inflammation) and/or to reduce B-cell autoantibody production within that mammal. For example, a therapeutically effective amount of an antimicrobial agent can be formulated together with one or more pharmaceutically acceptable carriers (additives) and/or diluents. A pharmaceutical composition can be formulated for administration in solid or liquid form including, without limitation, in the form of sterile solutions, suspensions, sustained-release formulations, tablets, capsules, pills, powders, or granules.

Pharmaceutically acceptable carriers, fillers, and vehicles that can be used in a pharmaceutical composition described herein can include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

A pharmaceutical composition containing one or more antimicrobial agents can be designed for oral or parenteral (including subcutaneous, intramuscular, intravenous, and intradermal) administration. When being administered orally, a pharmaceutical composition can be in the form of a pill, tablet, or capsule. Compositions suitable for parenteral administration can include aqueous and non-aqueous sterile injection solutions that can contain anti-oxidants, buffers, bacteriostats, and solutes that render the formulation isotonic with the blood of the intended recipient. The formulations can be presented in unit-dose or multi-dose containers, for example, sealed ampules and vials, and can be stored in a freeze dried (lyophilized) condition requiring the addition of the sterile liquid carrier, for example, water for injections, immediately prior to use. Extemporaneous injection solutions and suspensions can be prepared from sterile powders, granules, and tablets.

In some cases, a pharmaceutically acceptable composition including one or more antimicrobial agents can be administered locally or systemically. For example, a composition provided herein can be administered locally by intravenous injection or blood infusion. In some cases, a composition provided herein can be administered systemically, orally, or by injection to a mammal (e.g., a human).

The effective amount (e.g., effective dose) of a composition containing one or more antimicrobial agents can be any amount that reduces infection (and subsequent inflammation) or B-cell autoantibody production (e.g., B-cell antibody production inhibition or reduction in the number of B-cells) within a mammal having membranous nephropathy without producing significant toxicity to the mammal. For example, an effective amount of penicillin to treat membranous nephropathy as described herein can be about 2.4 million units (e.g., administered once by intramuscular injection). For example, an effective amount of doxycycline to treat membranous nephropathy as described herein can be about 100 mg (e.g., administered orally twice a day for about 14 days). For example, an effective amount of tetracycline to treat membranous nephropathy as described herein can be about 500 mg (e.g., administered orally four times a day for about 14 days). If a particular mammal fails to respond to a particular amount, then the amount of an antimicrobial agent can be increased by, for example, two fold. After receiving this higher amount, the mammal can be monitored for both responsiveness to the treatment and toxicity symptoms, and adjustments made accordingly. For example, levels of anti-NDNF autoantibodies present within the mammal (e.g., within the blood of the mammal) can be monitored by an appropriate method (e.g., ELISA). In some cases, the effective amount of a composition containing one or more antimicrobial agents can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition can require an increase or decrease in the actual effective amount administered.

The frequency of administration of one or more antimicrobial agents can be any amount that reduces infection (and subsequent inflammation) or B-cell autoantibody production (e.g., B-cell antibody production inhibition or reduction in the number of B-cells) within a mammal having membranous nephropathy without producing significant toxicity to the mammal. For example, the frequency of administration of an antimicrobial agent can be from about once a day to about four times a day (e.g., from about once a day to about four times a day, from about once a day to about three times a day, from about once a day to about twice a day, from about twice a day to about four times a day, from about three times a day to about four times a day, or from about twice a day to about three times a day). The frequency of administration of one or more antimicrobial agents can remain constant or can be variable during the duration of treatment. A course of treatment with a composition containing one or more antimicrobial agents can include rest periods. For example, a composition containing one or more antimicrobial agents can be administered daily over a two-week period followed by a two-week rest period, and such a regimen can be repeated multiple times. As with the effective amount, various factors can influence the actual frequency of administration used for a particular application. For example, the effective amount, duration of treatment, use of multiple treatment agents, route of administration, and severity of the condition may require an increase or decrease in administration frequency.

An effective duration for administering a composition containing one or more antimicrobial agents can be any duration that reduces infection (and subsequent inflammation) or B-cell autoantibody production (e.g., B-cell antibody production inhibition or reduction in the number of B-cells) within a mammal having membranous nephropathy without producing significant toxicity to the mammal. In some cases, the effective duration can vary from several days to several months. In general, the effective duration for administering a composition containing one or more antimicrobial agents to treat membranous nephropathy can range in duration from about one day to about one month (e.g., from about one day to about 14 days, from about one day to about 7 days, from about 7 days to about one month, from about 14 days to about one month, or from about 7 days to about 14 days). Multiple factors can influence the actual effective duration used for a particular treatment. For example, an effective duration can vary with the frequency of administration, effective amount, use of multiple treatment agents, route of administration, and severity of the condition being treated.

In some cases, one or more antimicrobial agents can be used as the sole active agent to treat a mammal (e.g., a human) having membranous nephropathy that is identified as having (a) autoantibodies specific for a NDNF polypeptide and/or (b) kidney tissue (e.g., GBM) having an elevated level of a NDNF polypeptide as described herein.

In some cases, one or more antimicrobial agents can be administered to a mammal (e.g., a human) having membranous nephropathy that is identified as having (a) autoantibodies specific for a NDNF polypeptide and/or (b) kidney tissue (e.g., GBM) having an elevated level of a NDNF polypeptide as described herein together with one or more (e.g., one, two, three, four, five or more) immunosuppressive agents. Any appropriate immunosuppressive agent can be administered to a mammal (e.g., a human that was identified as having (a) autoantibodies specific for a NDNF polypeptide and/or (b) kidney tissue (e.g., GBM) having an elevated level of a NDNF polypeptide as described herein) to treat membranous nephropathy. In some cases, an immunosuppressive agent used as described herein to treat membranous nephropathy can reduce inflammation and/or reduce B-cell autoantibody production within a mammal. Examples of immunosuppressive agents that can be used as described herein to treat membranous nephropathy include, without limitation, mycophenolate mofetil (e.g., Cellcept); steroids such as prednisone; B-cell inhibitors such as anti-CD20 antibodies (e.g., rituximab); and calcineurin inhibitors such as cyclosporine and tacrolimus. In cases where one or more antimicrobial agents are used in combination with one or more immunosuppressive agents, the one or more immunosuppressive agents can be administered at the same time (e.g., in a single composition containing antimicrobial agents and the one or more immunosuppressive agents) or independently. For example, one or more antimicrobial agents can be administered first, and the one or more immunosuppressive agents administered second, or vice versa.

In some cases, a course of treatment and/or the severity of one or more symptoms related to membranous nephropathy can be monitored. Any appropriate method can be used to determine whether or not membranous nephropathy is being treated. For example, immunological techniques (e.g., ELISA) can be performed to determine if the level of anti-NDNF autoantibodies present within a mammal being treated as described herein is reduced following the administration of one or more antimicrobial agents. Remission and relapse of the disease can be monitored by testing for one or more markers for membranous nephropathy. In some cases, remission can be ascertained by detecting the disappearance or reduction of autoantibodies having the ability to bind to a NDNF polypeptide in the sera. In some cases, relapse of membranous nephropathy can be ascertained by a reappearance or elevation of autoantibodies to having the ability to bind to a NDNF polypeptide in the sera.

The invention will be further described in the following examples, which do not limit the scope of the invention described in the claims.

### EXAMPLES

### Example 1: Membranous Nephropathy in Syphilis is Associated with Neuron-Derived Neurotrophic Factor

This Example identifies neuron-derived neurotrophic factor (NDNF) as a target antigen in syphilis-associated membranous nephropathy, and describes how NDNF can be used as a serological biomarker and/or therapeutic target of syphilis-associated membranous nephropathy.

### Methods

### Patients and Sample Collection

Biopsies for diagnosis were evaluated. The diagnosis of MN was confirmed by light microscopy (LM), immunofluorescence (IF) microscopy including PLA2R studies, and electron microscopy (EM). The clinical information was obtained from the accompanying charts. For detection of novel proteins, MS/MS was performed in 250 cases of PLA2R-negative cases (discovery cohort) that included cases used for identification of EXT1/EXT2, NELL1, SEMA3B, PCDH7, and FAT1 (Sethi et al., J. Am. Soc. Nephrol., 30:1123-1136 (2019); Sethi et al., Kid. Int., 97:163-174 (2020); Sethi et al., Kid. Int., 98:1253-1265 (2020); Sethi et al., J. Am. Soc. Nephrol., 32:1249-1261 (2021); and Sethi et al., J. Am. Soc. Nephrol., 33:1033-1044 (2022)). A NDNF polypeptide was detected in 3 cases of PLA2R-negative MN (NDNF-associated MN) by MS/MS. All 3 cases were negative for spectral counts for THSD7A, EXT1/EXT2, NELL1, SEMA3B, HTRA1, CNTN1, NCAM1, PCDH7, and FAT1 while baseline spectral counts for PLA2R were present in 2 of the 3 cases.

Of the 3 NDNF-associated MN from the discovery cohort, one patient had HIV (case 1), one had a lung tumor (case 2), and one had syphilis and hepatitis B co-infection (case 3). Using MS/MS, 14 cases of hepatitis B-associated MN and 5 cases of syphilis-associated MN were screened for MN antigens (validation cohort). Tissue from 4 cases (cases 4-7) of syphilis-associated MN were obtained from Cedars Sinai Medical Center and one case (cases 8-9) from Mayo Clinic. Cases 8 and 9 were from the same patient who was biopsied twice, 3 months apart. All cases were retrospective and retrieved from the tissue registry after review of the clinical charts for syphilis and membranous nephropathy. A case of infection-related glomerulonephritis in the setting of syphilis (case 10) was also retrieved.

For control cases, MS/MS was performed on 116 cases that included 15 cases of time zero kidney transplant biopsies, 17 cases of minimal change disease, 44 cases of focal segmental glomerulosclerosis, 7 cases of diabetic glomerulosclerosis, 5 cases of IgA nephropathy, and 28 cases of PLA2R-associated MN. The initial 250 PLA2R-negative MN and control cases had been characterized and previously utilized for MS/MS studies in the discovery of EXT1/EXT2, NELL1, SEMA3B, PCDH7, and FAT1 (see, e.g., Sethi et al., J. Am. Soc. Nephrol., 30:1123-1136 (2019); Sethi et al., Kid. Int., 97:163-174 (2020); Sethi et al., Kid. Int., 98:1253-1265 (2020); Sethi et al., J. Am. Soc. Nephrol., 32:1249-1261 (2021); and Sethi et al., J. Am. Soc. Nephrol., 33:1033-1044 (2022)). None of the control cases showed any spectral counts for NDNF.

### Protein Identification by Laser Capture Microdissection, Trypsin Digestion, Nano-LC Orbitrap Tandem Mass Spectrometry (MS/MS)

For each case, 10-micron thick formalin-fixed paraffin sections (FFPE) were obtained and mounted on a special PEN membrane laser microdissection slide and using a Zeiss Palm Microbean microscope, the glomeruli were microdissected to reach approximately 250-550000 μM² per case. Resulting FFPE fragments were digested with trypsin and collected for MS/MS analysis. The trypsin digested peptides were identified by nano-flow liquid chromatography electrospray tandem MS/MS (nanoLC-ESI-MS/MS) using a Thermo Scientific Q-Exactive or Exploris480 Mass Spectrometer (Thermo Fisher Scientific, Bremen, Germany) coupled to a Thermo Ultimate 3000 RSLCnano HPLC system. All MS/MS samples were analyzed using Mascot and X! Tandem set up to search a Swissprot human database. Scaffold (version 4.8.3, Proteome Software Inc., Portland, OR) was used to validate MS/MS based peptide and protein identifications. Peptide identifications were accepted at greater than 95.0% probability by the Scaffold Local FDR algorithm with protein identifications requiring a 2 peptide minimum and a 95% probability using Protein Prophet.

The glomerular areas dissected for each NDNF positive case were as follows: case 1-473352 μM²; case 22-156770 μM²; case 3-305966 μM²; case 4-622776 μM²; case 5-560798 μM²; case 6-370425 μM²; case 7-723687 μM²; case 8-984578 μM²; and case 9-1236777 μM².

### Immunohistochemical (IHC) Staining

Tissue sectioning and IHC staining was performed using a Leica Bond RX stainer (Leica). FFPE tissues were sectioned at 5 microns and IHC staining was performed on-line. Slides for NDNF stain underwent antigen retrieval for 20 minutes using Epitope Retrieval 2 (EDTA; Leica) and incubated in Protein Block (Dako) for five minutes. The NDNF (rabbit polyclonal, Abcam) antibody was diluted to 1:400 in Background Reducing Diluent (Dako) and incubated for 15 minutes. The detection system used was Polymer Refine Detection System (Leica). This system includes the hydrogen peroxidase block, post primary and polymer reagent, diaminobenzidine (DAB), and hematoxylin. Immunostaining visualization was achieved by incubating slides for 10 minutes in DAB and DAB buffer (1:19 mixture) from the Bond Polymer Refine Detection System. Slides were rinsed between steps with 1× Bond Wash Buffer (Leica), counterstained for five minutes using Schmidt hematoxylin and molecular biology grade water (1:1 mixture), and rinsed several times in 1× Bond wash buffer and distilled water. Once the immunochemistry process was completed, slides were removed from the stainer and rinsed in tap water for five minutes. Slides were dehydrated in increasing concentrations of ethyl alcohol and cleared in 3 changes of xylene prior to permanent cover slipping in xylene-based medium.

IF confocal staining was performed on 8-mm sections cut from Bouin-fixed (patient 1) paraffin embedded biopsy sample. Slides were deparaffinized and antigen retrieval was performed with Proteinase-K treatment for 5 minutes at room temperature. After quenching in 100 mM ammonium chloride for 20 minutes and blocking (5% normal donkey serum, 5% BSA in 0.1% Triton X-100, PBS) for 2 hours, slides were incubated with primary antibody at 4° C. overnight. The following day, the secondary antibody incubation along with AF647 anti-human IgG antibody was performed at room temperature for 2 hours. Washes using PBS with 0.05% Triton X-100 were performed between the incubations and slides were mounted using Vectashield Antifade mounting medium. Primary antibody against NDNF (catalog number LS-C168140; LifeSpan Biosciences), AF647 anti-human IgG antibody (catalog number ab201841; abcam) and secondary antibody donkey anti-rabbit AF555N (Thermo Scientific) were used. Microscope images (Zeiss LSM780) were acquired using a 203 lens (numerical aperture 0.3) and images were prepared using Photoshop. All exposure levels were identical within the groups. Confocal IF for validation cohort (cases 4-7) and control cases including a case of PLA2R-associated and EXT1/EXT2-associated MN was carried out using AF647 anti-human IgG antibody (catalog #A21445; Invitrogen) with the similar protocol. Microscope images (Zeiss LSM780) were acquired using a 40× lens (numerical aperture 1.2) and images were prepared using Photoshop. All exposure levels were identical within the groups.

Elution of IgG from Kidney Biopsy Specimens

IgG was acid-eluted from frozen (tissue remaining after immunofluorescence microscopy) kidney biopsy specimens. The eluate containing anti-NDNF IgG was obtained from the 3 patients with NDNF-associated MN that were pooled together. The eluate containing control IgG was obtained from 2 patients with PLA2R-associated MN. Serial cryostat sections of 4 μm thickness were mounted side by side on a glass slide. The slides were thawed, fixed with pre-chilled 100% acetone for 10 minutes at room temperature and then washed for 5 minutes with phosphate-buffered saline (0.01 M, phosphate, pH 7.2). The slide sections were covered with 0.2 mL of a 0.02 M citrate buffer (pH 3.2) and incubated overnight in a humid chamber at 4° C. The eluate was extracted with a calibrated syringe and neutralized with 0.4 M NaOH to a pH of 7.2.

### Western Blot Analysis

A recombinant protein corresponding to antigenic determinants in human NDNF (Gln20-Phe331, Abbexa©) was used under non-reducing and reducing conditions. The target molecular weight and dominant band is expected at 44 kDa. The protein (2000 ng) was diluted with non-reduced and reduced (with β-mercaptoethanol) Laemmli sample buffer (BioRad) and boiled for 5 minutes. The samples were loaded into Criterion 12% TGX gels (BioRad) and electrophoresed in Tris-glycine-SDS running buffer. Proteins were transferred to nitrocellulose membrane (0.20 μm pore size) according to standard protocols. Membranes were incubated overnight at 4° C. with rabbit anti-human NDNF (1:1000, recommended dilution Antibodies Online©) and eluates from NDNF-associated MN and PLA2R-associated MN. Subsequently, blots were washed and incubated 1 hour at room temperature with goat anti-rabbit and goat anti-human IgG Fc (1:15000 and 1:5000, respectively, LI-COR©). Near infrared fluorescence was detected at the 700 and 800 nm channels in the Odyssey Infrared Imaging System (LI-COR© Biosciences, Lincoln, NE).

### Results

### MS/MS Detection of NDNF in PLA2R-negative MN Biopsies

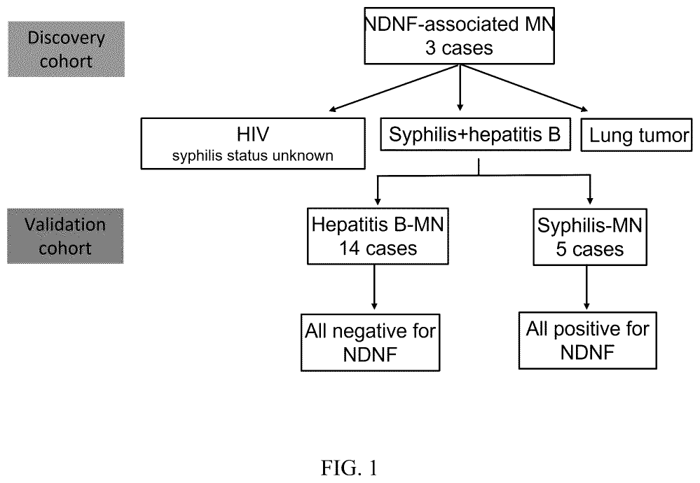

A unique protein, NDNF, was detected by MS/MS in the glomeruli of 8 cases of MN: 3 in the discovery cohort and 5 cases of validation cohort (FIG. 1). NDNF was present in all cases of syphilis-associated MN, and one case each of HIV and lung tumor (syphilis status was not known in these 2 patients). NDNF was absent in all cases of hepatitis B-associated MN.

The total spectral counts of NDNF ranged from 21-80 in the 8 cases with an average total spectral count of 44.1 (SD±18.4). The lowest total spectral counts were found in the patient with lung tumor. One patient was biopsied twice 3 months apart (first biopsy shown as case 8 and second biopsy shown as case 9) with similar MS/MS findings. MS/MS was also performed in a biopsy showing diffuse proliferative glomerulonephritis in a patient with active syphilis. The biopsy findings were consistent with an infection-associated glomerulonephritis. MS/MS in this case was negative for NDNF (shown as case 10 in FIG. 2A).

All control cases including 15-time 0 transplant biopsies, 73 other glomerulopathies, and 28 PLA2R-associated MN cases were negative for NDNF.

The spectral counts of 8 cases including the patient biopsied twice (case 8 and 9) of NDNF-positive MN along with a representative sequence coverage map of NDNF are shown in FIG. 2A-B. The MS/MS spectra match from one case is shown in FIG. 2C. None of the NDNF-positive cases show any spectral counts for EXT1/EXT2, THSD7A, NELL1, NCAM1, CNTN1, HTRA1, SEMA3B, PCDH7, or FAT1 while baseline PLA2R counts were detected in 7 of the 8 cases.

The main subclasses present in NDNF-associated MN were IgG1, IgG2 and IgG3, with average spectral counts of IgG1 66.1 (S.D±27.3), IgG2 42.4 (S.D±10.8), and IgG3 57.6 (S.D±13.1). IgG4 was the least abundant IgG with spectral counts of 21.1 (S.D±9.3). This is in keeping with the IgG subtypes detected on IF microscopy in cases 3 and 4.

### Immunohistochemical (IHC) and Confocal Immunofluorescence (IF) Staining:

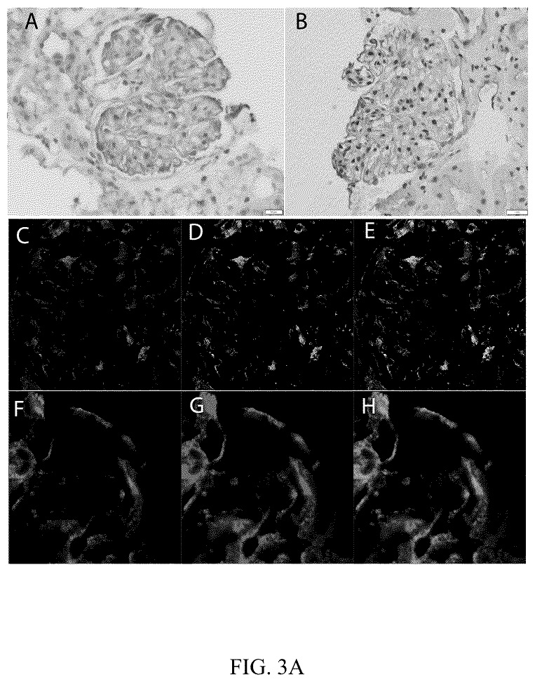

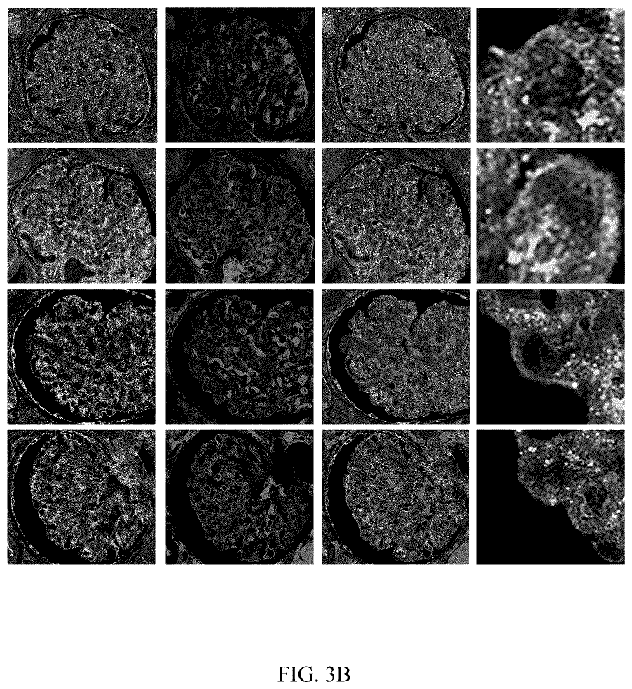

IHC staining was performed on biopsy samples from patients 2 and 3, and confocal IF staining was performed on biopsy sample from patient 3-7. IHC studies showed granular (2-3+) staining along the GBM in both cases (FIG. 3A, panels A-B).

Confocal IF microscopy was initially performed on case 1 to show that the NDNF and IgG co-localize along the GBM (FIG. 3A, panels C-H). Granular staining for NDNF (FIG. 3A, panels C and F) and IgG (FIG. 3A, panels D and G) is seen in along the GBM, and superimposition of the 2 figures (FIG. 3A, panels E and H) confirms the colocalization of NDNF and IgG. The co-localization of NDNF and IgG further corroborates that the subepithelial deposits contain both NDNF and IgG.

Additional confocal IF microscopy studies were performed on the validation cases (cases 4-7, rows 1-4) that show granular staining for NDNF (column 1), IgG (column 2), NDNF and IgG overlay (column 3) and high power of NDNF and IgG overlay in selected loops (column 4) (FIG. 3B). Interestingly, confocal IF microscopy showed positive granular mesangial deposits in addition to capillary walls deposits, which is in keeping with the mesangial deposits noted on EM studies. Control cases using PLA2R-associated MN and EXT 1/2-associated MN were negative for NDNF (bottom panel).

### IgG Elution and Western Blot Studies

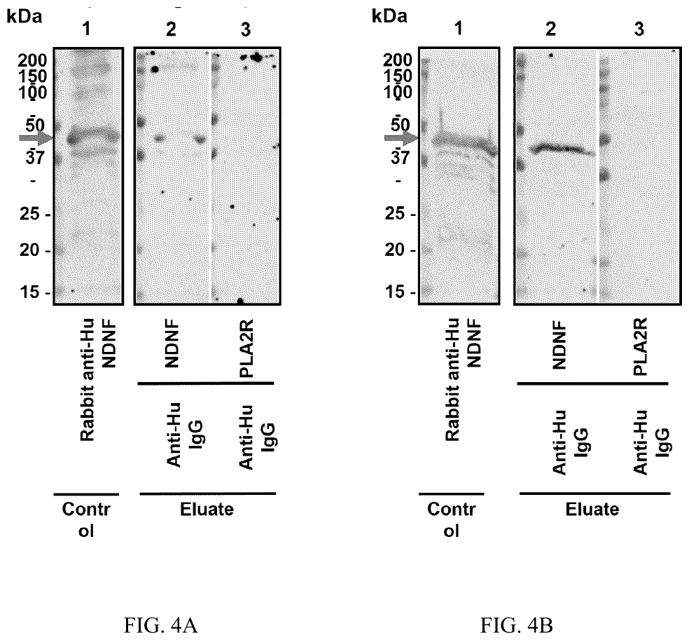

Western blot analyses were performed using recombinant human NDNF to determine the presence of anti-NDNF antibodies in the eluate obtained from pooled kidney biopsies of 3 NDNF-associated MN cases (cases 1-3, FIG. 4). NDNF was detected by rabbit anti-human NDNF under both non-reducing and reducing conditions. FIG. 4 shows a dominant band as expected at approximately 44 kDa to both non-reduced and reduced NDNF using rabbit anti-human NDNF (lane 1, positive control). The same band was detected after exposure of nonreduced and reduced human NDNF to IgG obtained from the eluate of NDNF-associated MN (lane 2). Anti-NDNF antibodies were not detected in IgG eluate obtained from patients with PLA2R-associated-MN (lane 3).

### Clinical and Laboratory Findings of NDNF-Associated MN

The mean age of NDNF-associated MN was 43.7±17 years (range 22-73, median 30). There were 6 males and 2 females. The mean serum creatinine and proteinuria at kidney biopsy was 2.1±2.4 mg/dl and 8.8±3.9 grams/day, respectively. Case 1 had HIV, case 2 had a lung tumor, and cases 3-8 had syphilis with positive RPR (rapid plasma reagin) test at the time of development of nephrotic syndrome. Clinical charts did not reveal whether cases 1 and 2 were tested for syphilis.

Kidney biopsy revealed a MN in all cases with thickened GBMs and no proliferative findings. Immunofluorescence findings revealed granular IgG3+ staining along the GBM in all cases but also with variable IgA and IgM (1-2+) in 5 of the 8 cases. Bright C3 (2-3+) was present in all cases. Clq (1+-3+) was also present in 7 of the 8 cases. There was no tubular basement membrane staining for IgG in any of the cases. Electron microscopy revealed subepithelial electron dense deposits in all cases. In many loops, the deposits appeared as subepithelial humps. The deposits were superficial, appeared as a line of subepithelial humps, and there was minimal extension of GBMs around deposits (stage I MN of Ehrenreich and Churg). Subendothelial deposits were absent. Few mesangial deposits were present in most cases. Acute tubular injury and acute interstitial nephritis were also noted in 3 cases. PLA2R staining was negative in all cases. IgG subtypes done in 2 cases showed IgG1 3+, IgG2 1-2+, and IgG3 2+ along the GBM, but no staining for IgG4.

Follow-up was available in 4 of the 6 cases of syphilis. All patients were treated with antibiotics (penicillin) with complete remission of the nephrotic syndrome. Case 8 achieved complete remission following treatment with penicillin yet, the biopsy continued to show membranous nephropathy in a repeat biopsy 3 months later. Following the diagnosis of membranous nephropathy in case 2, work up revealed a lung tumor. Following successful treatment (resection) of the lung tumor, the nephrotic syndrome went into complete remission.

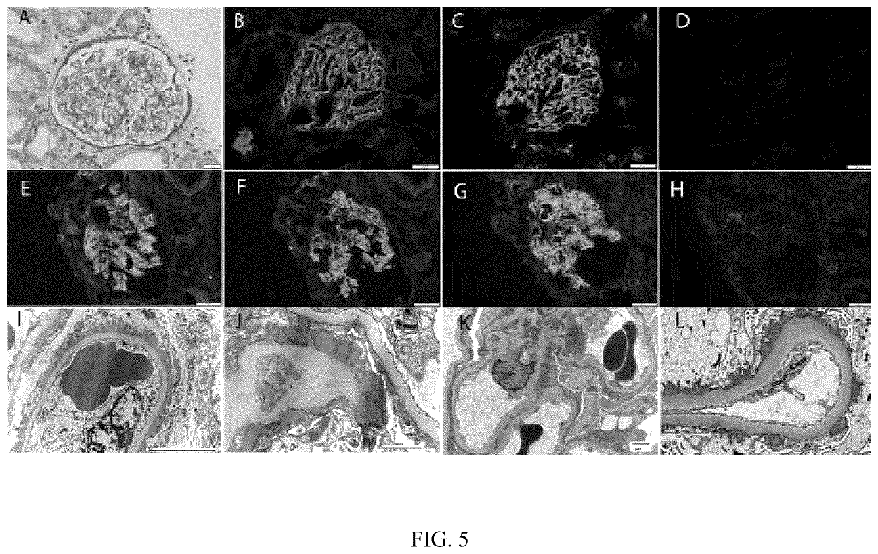

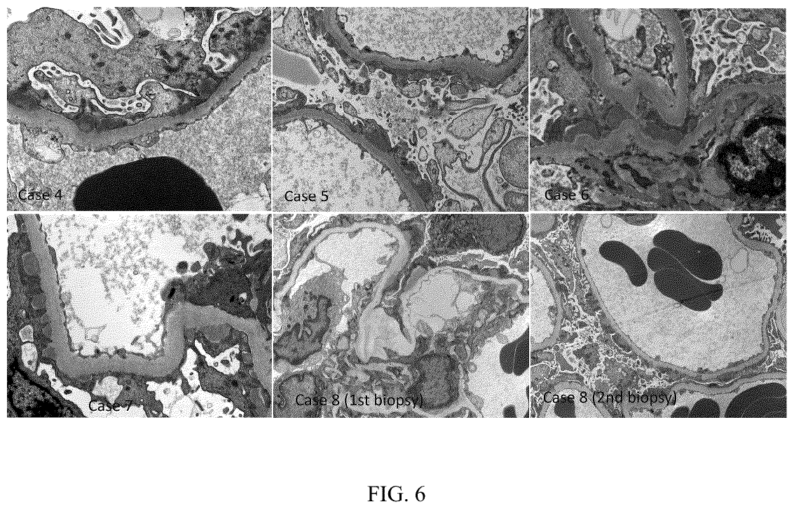

Representative biopsy findings of Case 3 are shown in FIG. 5 (panels A-H), and EM showing additional EMs form cases 1 and 2 are shown superficial hump-like deposits are also shown (FIG. 5, panels I-L). EM for cases 4-8 showing similar findings are shown in FIG. 6.

TABLE 1
Clinical and kidney biopsy findings.

| | | | Urinary | | | Total/ | | | | Hump-like | |
|---|---|---|---|---|---|---|---|---|---|---|---|
| | | Serum Cr | Protein | Clinical | | sclerosed | IFTA | | EM | subepithelial | Additional |
| Case | Age/Sex | mg/dL | (g/24 hours) | association | Follow-up | glomeruli | % | IF | stage | deposits | findings |
| 1 | 30/M | 0.9 | 9.7 | HIV/chronic | Lost to | 26/0 | 0 | IgG 2 + IgM 1 + | I | Yes | ATN, AIN |
| | | | | NSAID use | follow up | | | C1q 2 + C3 3 + | | | |
| | | | | | | | | No TBM IgG | | | |
| 2 | 73/M | 1.4 | 11 | Lung tumor | Complete | 12/3 | 20 | IgG 3 + C3 3 + | I | Yes | |
| | | | | | remission | | | No TBM IgG | | | |
| 3 | 56/M | 1.3 | 7.3 | Hepatitis B/ | Complete | 12/0 | 10 | IgG 3 +* C1q 2 + | I | Yes | |
| | | | | Syphilis | remission | | | C3 3 + | | | |
| | | | | | | | | No TBM IgG | | | |
| 4 | 24/F | 3.4 | 14 | Syphilis | Started | 20/0 | 0 | IgG 3 +* C1q 3 + | I | Yes | ATN |
| | | | | | penicillin, | | | C3 2 + | Mes | | |
| | | | | | Lost to | | | No TBM IgG | | | |
| | | | | | follow up | | | | | | |
| 5 | 22/M | 0.8 | 1.9 | Syphilis | No follow up | 34/0 | 5 | IgG 3 + IgM 1 + | I | Yes | |
| | | | | | | | | IgA 1 + C1q 1 + | Mes | | |
| | | | | | | | | C3 3 + | | | |
| | | | | | | | | No TBM IgG | | | |
| 6 | 40/F | 0.9 | 13 | Syphilis | Complete | 12/0 | 2 | IgG 3 + IgM 1 + | I | Yes | AIN + |
| | | | | | remission | | | C1q 2 + C3 3 + | Mes | | Treponema |
| | | | | | | | | No TBM IgG | | | by IHC |
| 7 | 53/M | 7.8 | 7.0 | Syphilis | Complete | 20/0 | 0 | IgG 3 + IgM 1 + | I | Yes | ATN |
| | | | | | remission | | | IgA 1 + C1q 1 + | Mes | | |
| | | | | | | | | C3 3 + | | | |
| | | | | | | | | No TBM IgG | | | |
| 8 and | 52/M | 1.0 | 6.5 | Syphilis | Complete | 16/0 | 0 | IgG 3 + IgM 2 + | I | Yes | |
| 9** | | 0.8 | 250 mgs | | remission | 37/0 | 0 | IgA 1 + C1q 3 + | Mes | | |
| | | | | | | | | C3 3 + | | | |
| | | | | | | | | No TBM IgG | | | |

*IgG subtypes show IgG1, IgG2 and IgG3, with negative IgG4,
**biopsied twice

Together, these results demonstrate that the NDNF polypeptide is the target antigen in a syphilis-associated membranous nephropathy. Accordingly, the presence of (a) autoantibodies specific for a NDNF polypeptide and/or (b) a GBM having an elevated level of a NDNF polypeptide can be used to identify a mammal (e.g., a human that has syphilis) as having a NDNF-positive membranous nephropathy. In some cases, a mammal (e.g., a human that has syphilis) identified as having a NDNF-positive membranous nephropathy can be treated by administering one or more antimicrobial agents to the mammal.

### Example 2: Identifying NDNF Polypeptide Positive Membranous Nephropathy

A blood sample (e.g., serum) is obtained from a human having membranous nephropathy. The obtained sample is examined for the presence of autoantibodies specific for a NDNF polypeptide.

If autoantibodies specific for a NDNF polypeptide are detected in the sample, as compared to a control level, then the human is classified as having a NDNF positive membranous nephropathy.

### Example 3: Treating NDNF Polypeptide Positive Membranous Nephropathy

A human identified as having autoantibodies specific for a NDNF polypeptide is administered one or more antimicrobial agents (e.g., one or more antibiotic agents).

The administered antimicrobial agent(s) can reduce infection (and subsequent inflammation) and/or B-cell autoantibody production.

The administered antimicrobial agent(s) can reduce the level of autoantibodies specific for a NDNF polypeptide present within the human.

### Example 4: Identifying NDNF Polypeptide Positive Membranous Nephropathy

A kidney tissue sample is obtained from a human having membranous nephropathy. The obtained sample is examined for an elevated level of a NDNF polypeptide.

If an elevated level of a NDNF polypeptide is detected in the sample, as compared to a control level, then the human is classified as having a NDNF polypeptide positive membranous nephropathy.

### Example 5: Treating NDNF Polypeptide Positive Membranous Nephropathy

A human identified as having an elevated level of a NDNF polypeptide in the GBM of the kidney is administered one or more antimicrobial agents (e.g., one or more antibiotic agents).

The administered antimicrobial agent(s) can reduce infection (and subsequent inflammation) and/or B-cell autoantibody production.

The administered antimicrobial agent(s) can reduce a level of autoantibodies specific for a NDNF polypeptide present within the human.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating a mammal having membranous nephropathy, wherein said method comprises:
(a) identifying a mammal as having membranous nephropathy comprising (i) autoantibodies specific for a NDNF polypeptide or (ii) kidney tissue comprising an elevated level of said NDNF polypeptide, and
(b) administering an antimicrobial agent to said mammal.

2. The method of claim 1, wherein said mammal is a human.

3. The method of claim 1, wherein said mammal is identified as having said membranous nephropathy comprising said autoantibodies.

4. The method of claim 1, wherein said mammal is identified as having said membranous nephropathy comprising said kidney tissue.

5. The method of claim 1, wherein said antimicrobial agent is an antibiotic agent.

6. The method of claim 5, wherein said antibiotic agent is selected from the group consisting of penicillin, doxycycline, tetracycline, and amoxicillin.

7. The method of claim 1, wherein said mammal is administered an immunosuppressive agent.

8. A method for treating a mammal having membranous nephropathy, wherein said method comprises administering an antimicrobial agent to a mammal identified as having membranous nephropathy comprising (i) autoantibodies specific for a NDNF polypeptide or (ii) kidney tissue comprising an elevated level of said NDNF polypeptide.

9. The method of claim 8, wherein said mammal is a human.

10. The method of claim 8, wherein said mammal was identified as having said membranous nephropathy comprising said autoantibodies.

11. The method of claim 8, wherein said mammal was identified as having said membranous nephropathy comprising said kidney tissue.

12. The method of claim 8, wherein said antimicrobial agent is an antibiotic agent.

13. The method of claim 12, wherein said antibiotic agent is selected from the group consisting of penicillin, doxycycline, tetracycline, and amoxicillin.

14. The method of claim 8, wherein said mammal is administered an immunosuppressive agent.

15. A method for treating a mammal having membranous nephropathy and kidney tissue comprising an elevated level of a NDNF polypeptide, wherein said method comprises administering an antimicrobial agent to said mammal.

16. The method of claim 15, wherein said mammal is a human.

17. The method of claim 15, wherein said mammal comprises autoantibodies specific for said polypeptide.

18. The method of claim 15, wherein said mammal was identified as having said kidney tissue.

19. The method of claim 15, wherein said antimicrobial agent is an antibiotic agent.

20. The method of claim 19, wherein said antibiotic agent is selected from the group consisting of penicillin, doxycycline, tetracycline, and amoxicillin.

21. The method of claim 15, wherein said mammal is administered an immunosuppressive agent.
